(19) United States
(12) Patent Application Publication
(10) Pub. No.: US 2021/0008195 A1
(43) Pub. Date: Jan. 14, 2021

(54) VACCINES AGAINST HENDRA AND NIPAH VIRUS INFECTION

(71) Applicant: Zoetis Services LLC, Parsippany, NJ (US)

(72) Inventors: Paul Joseph Dominowski, Kalamazoo, MI (US); Duncan M. Mwangi, Portage, MI (US); Dennis Lee Foss, Mattawan, MI (US); Sharath K. Rai, Portage, MI (US); John Morgan Hardham, Kalamazoo, MI (US)

(21) Appl. No.: 16/970,731

(22) PCT Filed: Dec. 18, 2018
(86) PCT No.: PCT/US2018/066145

§ 371 Date: Aug. 18, 2020

Related U.S. Application Data
(60) Provisional application No. 62/608,092, filed on Dec. 20, 2017.

(51) Int. Cl. A61K 39/155; A61K 39/39

(52) U.S. Cl. CPC A61K 39/155; A61K 2039/55561; A61K 39/39

(57) ABSTRACT

Disclosed is a method of protecting an animal in need thereof from Hendra or Nipah virus infection comprising administering to said animal a single dose of a vaccine, the vaccine comprising:an antigen component comprising a Hendra antigen or a Nipah antigen; and an adjuvant comprising oil, polycationic carrier and a CpG containing immunostimulatory oligonucleotide, wherein the vaccine is a W/O emulsion.

### FIELD OF THE INVENTION

This invention is generally in the field of animal vaccines against Hendra virus (HeV) and Nipah virus (NiV) infections.

### BACKGROUND

Paramyxoviruses such as HeV and NiV possess two major membrane-anchored glycoproteins in the envelope of the viral particle. One glycoprotein is required for virion attachment to receptors on host cells and is designated as either hemagglutinin-neuraminidase protein (HN) or hemagglutinin protein (H), and the other is glycoprotein (G), which has neither hemagglutination nor neuraminidase activities. The attachment glycoproteins are type II membrane proteins, where the molecule's amino (N) terminus is oriented toward the cytoplasm and the protein's carboxy (C) terminus is extracellular. The other major glycoprotein is the fusion (F) glycoprotein, which is a trimeric class I fusogenic envelope glycoprotein containing two heptad repeat (HR) regions and a hydrophobic fusion peptide. HeV and NiV infect cells through a pH-independent membrane fusion process into receptive host cells through the concerted action of their attachment G glycoprotein and F glycoprotein following receptor binding. The primary function of the HeV and NiV attachment G glycoprotein is to engage appropriate receptors on the surfaces of host cells, which for the majority of well-characterized paramyxoviruses are sialic acid moieties. The HeV and NiV G glycoproteins utilize the host cell protein receptors ephrin B2 and/or ephrin B3 and antibodies have been developed which block viral attachment by the G glycoprotein (WO2006137931, Bishop (2008) J. Virol. 82: 11398-11409). Further, vaccines have been developed which also use the G glycoprotein as a means for generating an immunoprotective response against HeV and NiV infection (WO2009117035).

There is presently one licensed vaccine for the prevention of infection or disease caused by Hendra virus (Equivac® HeV; Zoetis) approved for use in horses, although no licensed vaccine exists for preventing Nipah virus infection. Both Nipah virus and Hendra virus are United States, National Institute of Allergy and Infectious Disease, category C priority agents of biodefense concern. Further, as these viruses are zoonotic Biological Safety Level-4 agents (BSL-4), production of vaccines and/or diagnostics, with safety is very costly and difficult. The United States Department of Agriculture classifies both Nipah virus and Hendra virus as High-Consequence Foreign Animal Diseases.

### SUMMARY OF INVENTION

In the first aspect, the invention provides a method of protecting an animal in need thereof from Hendra or Nipah virus infection comprising administering to said animal a single dose of a vaccine, the vaccine comprising: an antigen component comprising a Hendra antigen or a Nipah antigen; and an adjuvant comprising oil, polycationic carrier and a CpG containing immunostimulatory oligonucleotide, wherein the vaccine is a W/O emulsion.

In certain embodiments, the animal is a porcine animal and the Nipah antigen comprises an amino acid sequence that is at least 95% (e.g., at least 98%) identical to SEQ ID NO: 11 or to the amino acids 71-602 thereof.

In certain embodiments, wherein the animal is an equine animal and the Hendra antigen comprises an amino acid sequence that is at least 95% (e.g., at least 98%) identical to SEQ ID NO: 12 or to the amino acids 73-604 thereof.

In further embodiments that can be combined with any of the embodiments described above, the oil is a non-metabolizable oil.

In further embodiments that can be combined with any of the embodiments described above, the polycationic carrier is DEAE dextran.

In further embodiments that can be combined with any of the embodiments described above, said single dose of vaccine has volume of about 0.125 ml to about 2 ml.

### BRIEF DESCRIPTION OF THE DRAWINGS

FIGS. 1 and 2 illustrate SEQ ID NOs: 11 and 12, which are G glycoproteins of Nipah and Hendra viruses, respectively.

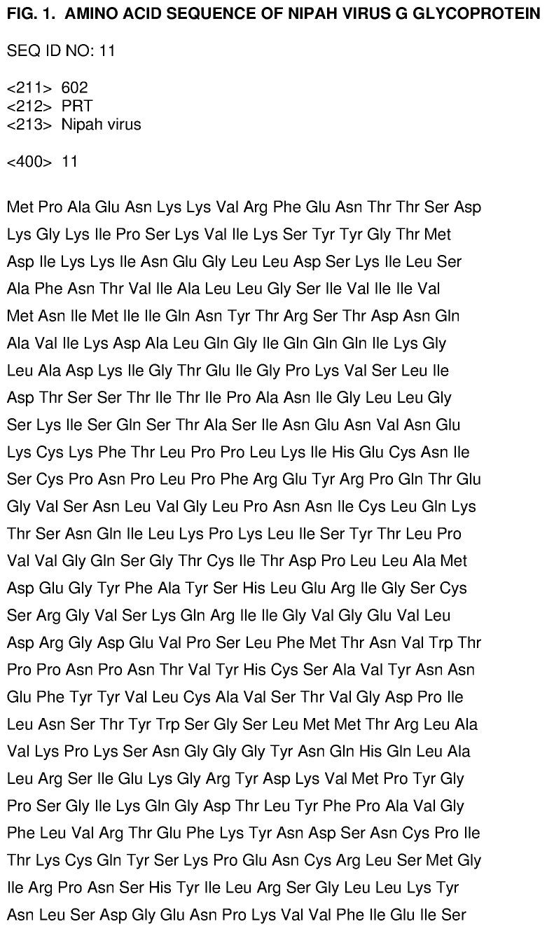

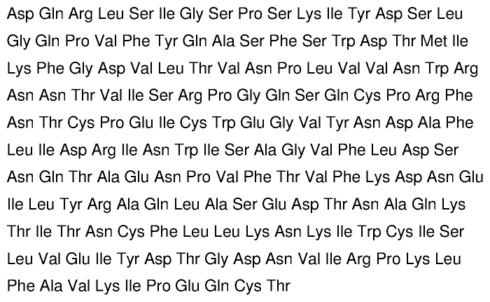

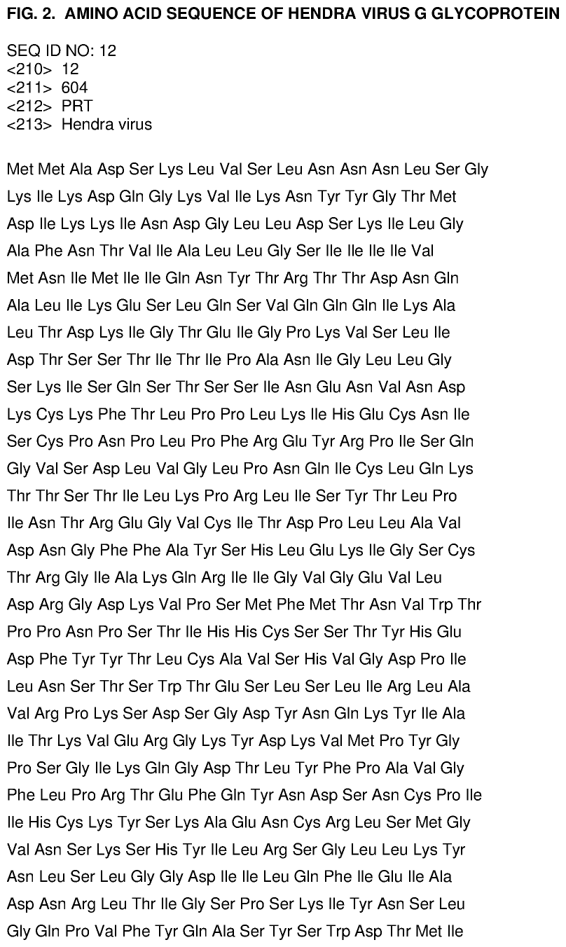

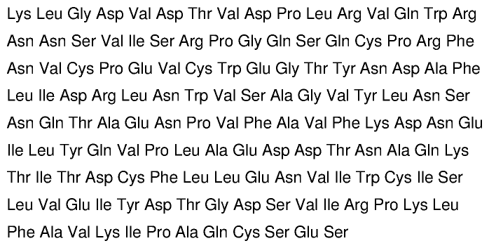

### DETAILED DESCRIPTION OF SELECTED EMBODIMENTS

Definitions

“About” or “approximately,” when used in connection with a measurable numerical variable, refers to the indicated value of the variable and to all values of the variable that are within the experimental error of the indicated value (e.g., within the 95% confidence interval for the mean) or within 10 percent of the indicated value, whichever is greater, unless about is used in reference to time intervals in weeks where “about 3 weeks,” is 17 to 25 days, and about 2 to about 4 weeks is 10 to 40 days.

“Antigen” or “immunogen” refers to any substance that is recognized by the animal's immune system and generates an immune response. The term includes killed, inactivated, attenuated, or modified live bacteria, viruses, or parasites. The term “antigen” also includes polynucleotides, polypeptides, recombinant proteins, synthetic peptides, protein extract, cells (including tumor cells), tissues, polysaccharides, or lipids, or fragments thereof, individually or in any combination thereof. The term antigen also includes antibodies, such as anti-idiotype antibodies or fragments thereof, and to synthetic peptide mimotopes that can mimic an antigen or antigenic determinant (epitope).

“Buffer” means a chemical system that prevents change in the concentration of another chemical substance, e.g., proton donor and acceptor systems serve as buffers preventing marked changes in hydrogen ion concentration (pH). A further example of a buffer is a solution containing a mixture of a weak acid and its salt (conjugate base) or a weak base and its salt (conjugate acid).

The method “comprising administering to a subject a single dose of vaccine X” excludes treatment regimens where more than one dose of vaccine X″ is administered.

“Consisting essentially” as applied to the adjuvant formulations refers to formulation which does not contain unrecited additional adjuvanting or immunomodulating agents in the amounts at which said agent exert measurable adjuvanting or immunomodulating effects.

The reference to a composition or vaccine being “effective as a single-dose vaccine” refers to a Nipah or Hendra vaccine which, upon a single administration to an animal that was not immunized against Nipah or Hendra, provides duration of immunity of at least five months, e.g., six months, seven months, eight months, nine months, ten months, eleven months, twelve months, thirteen months, or fourteen months against Nipah or Hendra challenge, respectively.

The term “emulsifier” is used broadly in the instant disclosure. It includes substances generally accepted as emulsifiers, e.g., different products of TWEEN® or SPAN® product lines (fatty acid esters of polyethoxylated sorbitol and fatty-acid-substituted sorbitan surfactants, respectively), and different solubility enhancers such as PEG-40 Castor Oil or another PEGylated hydrogenated oil.

“Immunologically protective amount” or “immunologically effective amount” or “effective amount to produce an immune response” of an antigen is an amount effective to induce an immunogenic response in the recipient. The immunogenic response may be sufficient for diagnostic purposes or other testing, or may be adequate to prevent signs or symptoms of disease, including adverse health effects or complications thereof, caused by infection with a disease agent. Either humoral immunity or cell-mediated immunity or both may be induced. The immunogenic response of an animal to an immunogenic composition may be evaluated, e.g., indirectly through measurement of antibody titers, lymphocyte proliferation assays, or directly through monitoring signs and symptoms after challenge with wild type strain, whereas the protective immunity conferred by a vaccine can be evaluated by measuring, e.g., reduction in clinical signs such as mortality, morbidity, temperature number, overall physical condition, and overall health and performance of the subject. The immune response may comprise, without limitation, induction of cellular and/or humoral immunity.

“Immunogenic” means evoking an immune or antigenic response. Thus an immunogenic composition would be any composition that induces an immune response.

“Lipids” refers to any of a group of organic compounds, including the fats, oils, waxes, sterols, and triglycerides that are normally considered insoluble (or sparingly soluble) in water but soluble in nonpolar organic solvents, are oily to the touch, and together with carbohydrates and proteins constitute the principal structural material of living cells.

“Pharmaceutically acceptable” refers to substances, which are within the scope of sound medical judgment, suitable for use in contact with the tissues of subjects without undue toxicity, irritation, allergic response, and the like, commensurate with a reasonable benefit-to-risk ratio, and effective for their intended use.

The present invention provides a method of vaccinating an animal in need thereof against Hendra and/or Nipah infection by administrating to the animal a single dose of the vaccine described herein. Briefly, the vaccine contains Hendra or Nipah antigen adjuvanted with adjuvant TXO as described in greater details below.

Antigen

Hendra virus G glycoprotein polypeptides that are useful in the practice of the present invention, and the recombinant expression thereof, reference is made to the entire disclosure of published international patent applications WO 2012/158643 and WO2006/085979 where such information is clearly set forth. Preferred examples of specific Hendra virus G protein polypeptides useful herein are disclosed in WO 2012/158643, and include, for example: full length G protein (SEQ ID NO: 12); a soluble fragment thereof (encoding amino acids 73-604 of SEQ ID NO: 12); and an additional fragment disclosed therein having an Ig(kappa) leader sequence. See, e.g., SEQ ID NO: 15 of WO 2012/158643. Generally, the soluble forms of the Hendra virus G glycoprotein comprises all or part of the ectodomain, and are produced by deleting all or part of the transmembrane domain of the G glycoprotein, and all or part of the cytoplasmic tail. Preferably, the encoding gene sequence is codon optimized for expression.

In some embodiments, the Hendra G glycoprotein may be in dimeric and/or tetrameric form. Such dimers depend upon the formation of disulfide bonds formed between cysteine residues in the G glycoprotein. Such disulfide bonds can correspond to those formed in the native G glycoprotein, or different disulfide bonds can be formed resulting in different dimeric and/or tetrameric forms of the G glycoprotein which enhance antigenicity. Additionally, non-dimerized and tetramerized forms are also useful according to the practice of the present invention, again taking into account that G glycoprotein provides numerous conformation-dependent epitopes (i.e. that arise from a tertiary three dimensional structure) and that preservation of numerous of such natural epitopes is accordingly highly preferred so as to impart a neutralizing antibody response.

Generally speaking, construction of expression vectors for the Hendra G proteins can be as described in Example 1 of WO 2012/158643, with resultant protein expression from CHO cells being as described in Example 2 thereof, or alternatively, using a Vaccinia system (see Example 3 thereof) or 293 cells (see Example 4 thereof). In a specific preferred example, the soluble G protein is provided as amino acids 73-604 of the native Hendra virus G glycoprotein (see SEQ ID NO: 2 in WO 2012/158643 which is identical to SEQ ID NO: 12. Dimerization thereof occurs spontaneously, concomitant with expression from CHO cells, and resultant G protein is approximately 50% dimer and 50% tetramer, with little remaining monomer. Expression in 293F cells leads to about 70% dimer.

Construction of expression vectors for Nipah G proteins has also been described. See, e.g., Examples 1 and 2 in WO 2012/158643. Preferred examples of specific Nipah virus G protein polypeptides useful herein are disclosed in WO 2012/158643, and include, for example: full length G protein (SEQ ID NO: 11); a soluble fragment thereof (encoding amino acids 71-602 of SEQ ID NO:11); and an additional fragment disclosed therein having an Ig(kappa) leader sequence. Generally, the soluble forms of the Hendra virus G glycoprotein comprises all or part of the ectodomain, and are produced by deleting all or part of the transmembrane domain of the G glycoprotein, and all or part of the cytoplasmic tail. Preferably, the encoding gene sequence is codon optimized for expression.

The Nipah G antigen may be produced similarly to Hendra G antigen, e.g., as described in Example 3 of WO 2012/158643.

Preferred doses of antigen for large animals are in the range of about 50 to about 200 micrograms per dose, with about 100 micrograms being a most preferred dose. For smaller animals, such as dogs, lesser amounts are needed, such as 5-50 micrograms, e.g., about 10 micrograms, about 15 micrograms, about 20 micrograms, about 25 micrograms, about 30 micrograms, about 35 micrograms, about 40 micrograms, about 45 micrograms.

In certain embodiments, the Nipah antigen and/or the Hendra antigen differ from SEQ ID NOs 11 and 12, respectively, by up to 5% of amino acid. Preferably, the altered amino acids are conservatively substituted. The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins, W. H. Freeman and Co., N. Y. (1984)).

In the embodiments, wherein the Hendra or Nipah antigen comprises an additional fragment (e.g., purification tags or Ig(kappa) leader sequence), in order to determine whether the antigen is at least 95% identical to SEQ ID NOs 11 or 12, such additional fragments are excluded from comparison.

In further embodiments, the antigen component may comprise a vector comprising a nucleic acid sequence that encodes any of the amino acid sequences described above. Suitable vectors include poxvectors (e.g., vaccinia vectors or canarypox vectors such as ALVAC), adenoviral vectors, SIRNAVAX℠ platform and the like.

Adjuvant

The vaccines of the instant invention are water-in-oil (W/O) emulsions. Multiple oils and combinations thereof are suitable for use of the instant invention. These oils include, without limitations, animal oils, vegetable oils, as well as non-metabolizable oils. Non-limiting examples of vegetable oils suitable in the instant invention are corn oil, peanut oil, soybean oil, coconut oil, and olive oil. Non-limiting example of animal oils is squalane. Suitable non-limiting examples of non-metabolizable oils include light mineral oil, straight chained or branched saturated oils, and the like.

In a set of embodiments, the oil used in the adjuvant formulations of the instant invention is a light mineral oil. As used herein, the term “mineral oil” refers to a mixture of liquid hydrocarbons obtained from petrolatum via a distillation technique. The term is synonymous with “liquefied paraffin”, “liquid petrolatum” and “white mineral oil.” The term is also intended to include “light mineral oil,” i.e., oil which is similarly obtained by distillation of petrolatum, but which has a slightly lower specific gravity than white mineral oil. See, e.g., Remington's Pharmaceutical Sciences, 18th Edition (Easton, Pa.: Mack Publishing Company, 1990, at pages 788 and 1323). Mineral oil can be obtained from various commercial sources, for example, J. T. Baker (Phillipsburg, Pa.), USB Corporation (Cleveland, Ohio). Preferred mineral oil is light mineral oil commercially available under the name DRAKEOL®.

Typically, the oily phase is present in an amount from 50% to 95% by volume; preferably, in an amount of greater than 50% to 85%; more preferably, in an amount from greater than 50% to 60%, and more preferably in the amount of greater than 50-52% v/v of the vaccine composition. The oily phase includes oil and emulsifiers (e.g., SPAN® 80, TWEEN® 80 etc), if any such emulsifiers are present. The volume of the oily phase is calculated as a sum of volumes of the oil and the emulsifier(s). Thus, for example, if the volume of the oil is 40% and the volume of the emulsifier(s) is 12% of a composition, then the oily phase would be present at 52% v/v of the composition. Similarly, if the oil is present in the amount of about 45% and the emulsifier(s) is present in the amount of about 6% of a composition, then the oily phase is present at about 51% v/v of the composition.

In a subset of embodiments, applicable to all adjuvants/vaccines of the instant invention, the volume percentage of the oil and the oil-soluble emulsifier together is at least 50%, e.g., 50% to 95% by volume; preferably, in an amount of greater than 50% to 85%; more preferably, in an amount from 50% to 60%, and more preferably in the amount of 50-52% v/v of the vaccine composition. Thus, for example and without limitations, the oil may be present in the amount of 45% and the lipid-soluble emulsifier would be present in the amount of greater than 5% v/v. Thus, the volume percentage of the oil and the oil-soluble emulsifier together would be at least 50%.

In yet another subset, applicable to all vaccines of the invention, volume percentage of the oil is over 40%, e.g., 40% to 90% by volume; 40% to 85%; 43% to 60%, 44-50% v/v of the vaccine composition.

Emulsifiers suitable for use in the present emulsions include natural biologically compatible emulsifiers and non-natural synthetic surfactants. Biologically compatible emulsifiers include phospholipid compounds or a mixture of phospholipids. Preferred phospholipids are phosphatidylcholines (lecithin), such as soy or egg lecithin. Lecithin can be obtained as a mixture of phosphatides and triglycerides by water-washing crude vegetable oils, and separating and drying the resulting hydrated gums. A refined product can be obtained by fractionating the mixture for acetone insoluble phospholipids and glycolipids remaining after removal of the triglycerides and vegetable oil by acetone washing. Alternatively, lecithin can be obtained from various commercial sources. Other suitable phospholipids include phosphatidylglycerol, phosphatidylinositol, phosphatidylserine, phosphatidic acid, cardiolipin, and phosphatidylethanolamine. The phospholipids may be isolated from natural sources or conventionally synthesized.

In additional embodiments, the emulsifiers used herein do not include lecithin, or use lecithin in an amount which is not immunologically effective.

Non-natural, synthetic emulsifiers suitable for use in the adjuvant formulations of the present invention include sorbitan-based non-ionic surfactants, e.g. fatty-acid-substituted sorbitan surfactants (commercially available under the name SPAN® or ARLACEL®), fatty acid esters of polyethoxylated sorbitol (TWEEN®), polyethylene glycol esters of fatty acids from sources such as castor oil (EMULFOR®); polyethoxylated fatty acid (e.g., stearic acid available under the name SIMULSOL® M-53), polyethoxylated isooctylphenol/formaldehyde polymer (TYLOXAPOL®), polyoxyethylene fatty alcohol ethers (BRIJ®); polyoxyethylene nonphenyl ethers (TRITON® N), polyoxyethylene isooctylphenyl ethers (TRITON® X). Preferred synthetic surfactants are the surfactants available under the name SPAN® and TWEEN®, such as TWEEN®-80 (Polyoxyethylene (20) sorbitan monooleate) and SPAN®-80 (sorbitan monooleate).

Generally speaking, the emulsifier(s) may be present in the vaccine composition in an amount of 0.01% to 40% by volume, preferably, 0.1% to 15%, more preferably 2% to 10%.

Additional ingredients present in the instant adjuvant formulations include cationic carriers and immunostimulatory oligonucleotides containing CpG. Such adjuvants forming W/O vaccine compositions comprising the immunostimulatory oligonucleotide and the polycationic carrier are referred to as “TXO”.

Suitable cationic carriers include, without limitations, dextran, DEAE (diethyl-aminoethyl) dextran (and derivatives thereof), PEGs, guar gums, chitosan derivatives, polycellulose derivatives like hydroxyethyl cellulose (HEC) polyethylenimene, poly aminos like polylysine and the like.

CpG oligonucleotides are a class of pharmacotherapeutic agents that are characterized by the presence of an unmethylated CG dinucleotide in specific base-sequence contexts (CpG motif). (Hansel T T, Barnes P J (eds): New Drugs for Asthma, Allergy and COPD. Prog Respir Res. Basel, Karger, 2001, vol 31, pp 229-232, which is incorporated herein by reference). These CpG motifs are not seen in eukaryotic DNA, in which CG dinucleotides are suppressed and, when present, usually methylated, but are present in bacterial DNA to which they confer immunostimulatory properties.

In selected embodiments, the adjuvants of the instant invention utilize a so-called P-class immunostimulatory oligonucleotide, more preferably, modified P-class immunostimulatory oligonucleotides, even more preferably, E-modified P-class oligonucleotides. P-class immunostimulatory oligonucleotides are CpG oligonucleotides characterized by the presence of palindromes, generally 6-20 nucleotides long. The P-Class oligonucleotides have the ability to spontaneously self-assemble into concatamers either in vitro and/or in vivo. These oligonucleotides are, in a strict sense, single-stranded, but the presence of palindromes allows for formation of concatamers or possibly stem-and-loop structures, as well as secondary and tertiary structures. The overall length of P- class immunostimulatory oligonucleotides is between 19 and 100 nucleotides, e.g., 19-30 nucleotides, 30-40 nucleotides, 40-50 nucleotides, 50-60 nucleotides, 60-70 nucleotides, 70-80 nucleotides, 80-90 nucleotides, 90-100 nucleotides.

In one aspect of the invention the immunostimulatory oligonucleotide contains a 5′ TLR activation domain and at least two palindromic regions, one palindromic region being a 5′ palindromic region of at least 6 nucleotides in length and connected to a 3′ palindromic region of at least 8 nucleotides in length either directly or through a spacer.

The P-class immunostimulatory oligonucleotides may be modified according to techniques known in the art. For example, J-modification refers to iodo-modified nucleotides. E-modification refers to ethyl-modified nucleotide(s). Thus, E-modified P-class immunostimulatory oligonucleotides are P-class immunostimulatory oligonucleotides, wherein at least one nucleotide (preferably 5′ nucleotide) is ethylated. Additional modifications include attachment of 6-nitro-benzimidazol, O-Methylation, modification with proynyl-dU, inosine modification, 2-bromovinyl attachment (preferably to uridine).

The P-class immunostimulatory oligonucleotides may also contain a modified internucleotide linkage including, without limitations, phosphodiesther linkages and phosphorothioate linkages. The oligonucleotides of the instant invention may be synthesized or obtained from commercial sources.

P-Class oligonucleotides and modified P-class oligonucleotides are further disclosed in published PCT application no. WO2008/068638, published on Jun. 12, 2008. Suitable non-limiting examples of modified P-class immunostiumulatory oligonucleotides are provided below (In SEQ ID NOs 1-10, “*” refers to a phosphorothioate bond and “_” refers to a phosphodiester bond). In SEQ ID NOs 11-14, all bonds are phosphodiester bonds.

SEQ ID NO: 1
5′ T*C_G*T*C_G*A*C_G*A*T*C_G*G*C*G*C_G*C*G*C*C*G 3′
SEQ ID NO: 2
5′ T*C_G*A*C*G*T*C*G*A*T*C*G*G*C*G*C*G*C*G*C*C*G 3′
SEQ ID NO: 3
5′ T*C*G*A*C*G*T*C*G*A*T*C*G*G*C*G*C*G*C*G*C*C*G*T
3′
SEQ ID NO: 4
5′ JU*C_G*A*C*G*T*C*G*A*T*C*G*G*C*G*C*G*C*G*C*C*G
3′
SEQ ID NO: 5
5′ JU*C_G*A*C*G*T*C*G*A*T*C*G*G*C*G*C*G*C*G*C*C*
G*T 3′
SEQ ID NO: 6
5′ JU*C*G*A*C*G*T*C*G*A*T*C*G*G*C*G*C*G*C*G*C*C*
G*T 3′
SEQ ID NO: 7
5′ EU*C_G*A*C*G*T*C*G*A*T*C*G*G*C*G*C*G*C*G*C*C*G
3′
SEQ ID NO: 8
5′ JU*C_G*T*C*G*A*C*G*A*T*C*G*G*C*G*G*C*C*G*C*C*
G*T 3′
SEQ ID NO: 9
5′ JU*C*G*T*C*G*A*C*G*A*T*C*G*G*C*G*G*C*C*G*C*C*
G*T 3′
SEQ ID NO: 10
5′ T*C_G*T*C_G*A*C_G*A*T*C_G*G*C*G*C_G*C*G*C*C*G 3′

In TXO adjuvants, the immunostimulatory oligonucleotide, preferably an ODN, preferably containing a palindromic sequence, and optionally with a modified backbone, may be present in the amount of 0.5-400 μg per dose, and the polycationic carrier may be present in the amount of 0.5-400 mg per dose. The dosages wary depending on the subject species.

For example, in certain embodiments more suitable for adult swine, one dose of TXO would comprise between about 50 and 400 μg (e.g., 50-300, or 100-250 μg, or about 50 to about 100 μg for adult pigs) of the immunostimulatory oligonucleotide, and the polycationic carrier may be present in the amount of between about 5 and about 500 mg per dose (e.g., 10-500 mg, or 10-300 mg, or 50-200 mg per dose). In embodiments more suitable for piglets, one dose of TXO would comprise between about 5 and 100 μg (e.g., 10-80 μg, or 20-50 μg) of the immunostimulatory oligonucleotide, while the polycationic carrier may be present in the amount of 1-50 mg per dose (e.g., 1-25 mg per dose, or 10-25 mg per dose).

TXO adjuvants may be prepared as follows:
a) Sorbitan monooleate is dissolved in light mineral oil. The resulting oil solution is sterile filtered;
b) The immunostimulatory oligonucleotide, DEAE Dextran and Polyoxyethylene (20) sorbitan monooleate are dissolved in aqueous phase, thus forming the aqueous solution; and
c) The aqueous solution is added to the oil solution under continuous homogenization thus forming the adjuvant formulation TXO.

The antigen may be added at the step of preparation of the aqueous phase—into the admixture of the immunostimulatory oligonucleotide and the polycationic carrier.

The vaccine may further comprise additional immunomodulatory molecules including without limitations, saponins (e.g., Quil A or purified fractions thereof), glycolipids, e.g.., BAY®R1005 (whether in a salt or a base form), MPLA, sterols (e.g., cholesterol), cationized sterols (e.g., 3β-[N-(N′,N′-dimethylaminoethane)-carbamoyl]cholesterol, also known as DC-Cholesterol), phospholipids (e.g., lecithin), alum, quaternary amines, e.g., DDA (dimethyl dioctadecyl ammonium) and the like.

The vaccine may further comprise different pharmaceutically acceptable excipients, e.g, buffers, pH and/or osmolarity adjusters, and/or preservatives. For example, Chlorocresol can be used as a preservative, in the amount of 0.01 to 0.5% w/v per dose, more preferably 0.05 to 0.2%, most preferably, about 0.1%. Other suitable excipients include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v), and combinations thereof.

Parenteral formulations are typically aqueous solutions which can contain excipients such as salts, carbohydrates and buffering agents (preferably to a pH of from about 3 to about 9, or from about 4 to about 8, or from about 5 to about 7.5, or from about 6 to about 7.5, or about 7 to about 7.5), but, for some applications, they can be more suitably formulated as a sterile non-aqueous solution or as a dried form to be used in conjunction with a suitable vehicle such as sterile, pyrogen-free water.

The preparation of parenteral formulations under sterile conditions, for example, by lyophilization, can readily be accomplished using standard pharmaceutical techniques well known to those skilled in the art.

The volume of the vaccine can be varied. Generally, standard dose for swine is about 2 ml of the vaccine per administration. In different embodiments, the volume can be varied, e.g., from 0.125 ml to about 5 ml, e.g., 2 ml, 1 ml, 0.5, ml, 0.25 ml etc. The decreased volume would still be a water-in-oil emulsion, preferably containing 50% of more of oily phase. The amounts of the antigen, the polycationic carrier and a CpG containing immunostimulatory oligonucleotide would preferably be the same as in the standard 2 ml dose. Such microdosing is advantageous in at least two aspects. First, it is less painful for an animal and second, particularly important for livestock animals, decreased amount of oil would metabolize faster and thus decrease slaughter withhold (i.e., the time between the vaccination and slaughter allowed by regulatory agencies).

Currently, there are no vaccines on the market containing Nipah antigen and only one vaccine containing Hendra antigen. EQUIVAC® Hendra (Zoetis) contains an antigen derived from Hendra G protein and is adjuvanted with ISCOMs (immunostimulating complex). EQUIVAC® Hendra is administered intramuscularly. Proper treatment regimen requires both prime and boost administrations (with boost administration about three weeks after the prime administration), and annual revaccinations. In contrast, the vaccines described herein are administered only once (as opposed to prime and boost administration) with annual revaccinations.

All publications cited in the specification, both patent publications and non-patent publications, are indicative of the level of skill of those skilled in the art to which this invention pertains. All these publications are herein fully incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

1. A method of protecting an animal in need thereof from Hendra or Nipah virus infection comprising administering to said animal a single dose of a vaccine, the vaccine comprising:
a) an antigen component comprising a Hendra antigen or a Nipah antigen; and
b) an adjuvant comprising oil, polycationic carrier and a CpG containing immunostimulatory oligonucleotide, wherein the vaccine is a W/O emulsion.

2. The method of claim 1, wherein the animal is a porcine animal and the Nipah antigen comprises an amino acid sequence that is at least 95% identical to SEQ ID NO: 11 or to the amino acids 71-602 thereof.

3. The method of claim 2, wherein the amino acid sequence is at least 98% identical to SEQ ID NO: 11 or to the amino acids 71-602 thereof.

4. The method of claim 1, wherein the animal is an equine animal and the Hendra antigen comprises an amino acid sequence that is at least 95% identical to SEQ ID NO: 12 or to the amino acids 73-604 thereof.

5. The method of claim 4, wherein the amino acid sequence is at least 98% identical to SEQ ID NO: 12 or to the amino acids 73-604 thereof.

6. The method of claim 1, wherein the oil is a non-metabolizabe oil.

7. The method of claim 6, wherein the non-metabolizable oil is light mineral oil.

8. The method of claim 1, wherein the polycationic carrier is selected from dextran, DEAE dextran, PEGs, guar gums, chitosan derivatives, polycellulose derivatives like hydroxyethyl cellulose (HEC) polyethylenimene, poly aminos and polylysine.

9. The method of claim 8 wherein the polycationic carrier is DEAE Dextran.

10. The method of claim 1, wherein said animal has not been previously vaccinated against Hendra or Nipah virus.

11. The method according to claim 1, wherein said single dose of vaccine has volume of about 0.125 ml to about 2 ml.

12. The method according to claim 11, wherein said single dose of vaccine has volume of about 0.25 ml to about 1 ml.
